(19) United States
(12) Patent Application Publication
(10) Pub. No.: US 2023/0202947 A1
(43) Pub. Date: Jun. 29, 2023

(54) Process and Plant for Producing Propylene

(71) Applicant: LINDE GMBH, Pullach (DE)

(72) Inventors: Ernst Haidegger, Riemerling (DE); Andreas Meiswinkel, Rimsting (DE); Hans-Jorg Zander, Munchen (DE)

(21) Appl. No.: 18/001,055

(22) PCT Filed: Jun. 8, 2021
(86) PCT No.: PCT/EP2021/065342

§ 371 Date: Dec. 7, 2022

(30) Foreign Application Priority Data
Jun. 10, 2020 (EP) 20179366.8

(51) Int. Cl. C07C 1/24; C07C 45/50

(52) U.S. Cl. CPC C07C 1/24; C07C 45/50

(57) ABSTRACT

A process for producing propylene in which a first material stream is provided using a steam cracking method and one or more fractionations and is rich in ethylene, in which a second material stream containing carbon monoxide and hydrogen is provided using a synthesis gas production method, and in which at least a part of the ethylene from the first material stream is reacted with at least a part of the carbon monoxide and the hydrogen from the second material stream to form an aldehyde using a hydroformylation to obtain a third material stream, and in which at least a part of the aldehyde in the third material stream is converted to the propylene, wherein the ethylene is provided by means of the steam cracking method in a first component mixture, wherein the propylene is provided in a second component mixture.

### CROSS-REFERENCE TO RELATED APPLICATIONS

This application is the national phase of, and claims priority to, International Application No. PCT/EP2021/065342, filed Jun. 8, 2021, which claims priority to European Application No. 20179366.8, filed Jun. 10, 2020, the disclosure of each of which is incorporated by reference herein in its entirety.

### FIELD OF THE INVENTION

The invention relates to a process for producing propylene and to a corresponding installation according to the preambles of the independent claims.

### BACKGROUND OF THE INVENTION

The production of propylene (propene) is described in the specialist literature, for example in the article “Propylene” in Ullmann's Encyclopedia of Industrial Chemistry, ed. 2012. Propylene is conventionally produced by steam-cracking hydrocarbon feedstocks and by conversion processes in the course of refinery processes. In the latter processes, propylene is not necessarily formed in the desired quantity and only as one of several components in a mixture with further compounds. Other processes for producing propylene are also known, but are not satisfactory in all cases, for example in terms of efficiency and yield.

An increasing demand for propylene (“propylene gap”), which requires the provision of corresponding selective methods, is predicted for the future. At the same time, it is necessary to reduce or completely prevent carbon dioxide emissions. As a potential starting compound, on the other hand, large amounts of methane are available, which are currently only fed to a material utilization in a very limited manner and are predominantly burned. In addition, appreciable amounts of ethane are often present in corresponding natural gas fractions.

An object of the invention is to provide an improved method, in particular in view of these aspects, for producing propylene.

### SUMMARY

Against this background, the invention proposes a process for producing propylene and a corresponding installation having the respective features of the independent claims. Preferred embodiments of the invention are the subject matter of the dependent claims and of the description provided herein.

### BRIEF DESCRIPTION OF THE DRAWINGS

FIG. 1 illustrates a process according to a preferred embodiment of the invention in the form of a schematic flowchart.

FIG. 2 illustrates a process according to a particularly preferred embodiment of the invention in the form of a schematic flowchart.

FIG. 3 illustrates a process according to a further particularly preferred embodiment of the invention in the form of a schematic flowchart.

### WRITTEN DESCRIPTION

In principle, in addition to the aforementioned steam cracking methods, a plurality of different methods exist for converting hydrocarbons and related compounds into one another, some of which will be mentioned below by way of example.

For example, the conversion of paraffins to olefins of identical chain length by oxidative dehydrogenation (ODH, also referred to as ODHE in the case of ethane) is known. Typically, a carboxylic acid of identical chain length is also formed as a coupling product in ODH, i.e. acetic acid in ODHE. However, ethylene can also be produced by the oxidative coupling of methane (OCM). Via a chain extension, for example by the hydroformylation described below, it is also possible to arrive at propylene starting from the ethylene formed in the oxidative dehydrogenation of ethane.

The production of propylene from propane by dehydrogenation (PDH) is also known and represents a commercially available and established process. The same also applies to the production of propylene from ethylene by olefin metathesis. This process requires 2-butene as an additional reagent.

Lastly, so-called methane-to-olefins or methane-to-propylenes (MTO, MTP) processes exist in which synthesis gas is first produced from methane and the synthesis gas is then reacted to give olefins, such as ethylene and propylene. Corresponding processes can be operated on the basis of methane, but also on the basis of other hydrocarbons or carbon-containing starting materials, such as coal or biomass.

Steam reforming and dry reforming as well as modifications thereof, including a downstream water gas shift for adjusting the ratio of hydrogen to carbon monoxide, are likewise known as individual technologies.

For synthesis gas production, partial oxidation (PDX), in particular with a higher proportion of carbon monoxide, is also known. Synthesis gases can also be produced by gasification of various feedstocks (coal, oil, waste, in particular plastic waste, etc.).

The hydroformylation already mentioned above in conjunction with the oxidative dehydrogenation of ethane represents a further technology which is used in particular for the production of so-called oxo compounds. Propylene is typically converted in the hydroformylation, but higher hydrocarbons, in particular hydrocarbons having six to eleven carbon atoms, or ethylene can also be used. Various processes for hydrogenating aldehydes and dehydrogenating alcohols (in particular ethanol and propanol) are likewise known.

For example, for the production of propylene, as mentioned, a hydroformylation can be used after an oxidative dehydrogenation of ethane. In the product stream of the oxidative dehydrogenation, certain fractions of carbon monoxide and especially unconverted ethane are present, inter alia. In particular, the high proportion of ethane in the range from 25 to 70 mole percent, in particular from 30 to 50 mole percent, leads to a significant dilution and significantly influences the partial pressures in the hydroformylation. Furthermore, carbon dioxide is formed in the oxidative dehydrogenation of ethane and must be separated off and, as necessary, treated separately. Dry reforming can be used for this purpose, for example. A significant integration of the purification and decomposition steps has not been known until now and the method steps taken into consideration hitherto are limited to the special case of an oxidative dehydrogenation of ethane and the use of a product stream which is not yet purified and the utilization of the by-product carbon dioxide in dry reforming.

Propylene can also be produced in principle starting from a component mixture which, in addition to ethylene, also contains considerable proportions of methane, carbon monoxide and carbon dioxide and optionally also hydrogen and ethane, and which can be provided, for example, by an oxidative coupling of methane. Here too, the further processing can comprise a hydroformylation. Here too, however, there is a significant dilution of the reactants which significantly influence the partial pressures in the hydroformylation. A typical ethylene content in a product mixture from the oxidative coupling of methane is basically rather at the lower end of a range of from 5 to 60 mole percent. The hydroformylation is preferably performed at high pressure, and in particular methane acts here as an inert medium which negatively influences the partial pressures of the reactants ethylene, carbon monoxide and hydrogen. Moreover, the comparatively high dilution also has a negative effect on the size of the apparatuses and thus makes the investment and operating costs more expensive. Corresponding disadvantages can also occur, for example, in principle by the formation of carbon dioxide when a shift reaction for converting carbon monoxide into hydrogen is undertaken.

Accordingly, U.S. Pat. No. 10,519,087 B2 also describes a method in which a feedstock stream from an oxidative coupling of methane is used, and thus contains, as explained, as well as ethylene, significant amounts of other components, in particular light and/or inert components, which then have to be conducted through the hydroformylation. Reference is made to the disadvantages just explained.

The above-mentioned methods all use a crude gas stream which, in addition to the components carbon monoxide, hydrogen and ethylene, also contains further components. A corresponding crude gas stream does not necessarily contain the correct composition for the subsequent method steps and can typically also contain inert components such as methane, ethane and optionally carbon dioxide, but also disruptive compounds, such as acetylene, oxygenates, etc. Therefore, a not inconsiderable purification and separation outlay is typically required in such methods.

The subject invention creates a method which makes it possible to contribute to covering the increasing demand for propylene mentioned at the outset, in particular also in relation to ethylene. In particular, it allows an integration of methane or other low-cost carbon sources into the petrochemical value chain in an advantageous manner compared to the prior art and the explained methods likewise possible in principle. This makes it possible, in particular, to utilize previously unused accompanying gases from petroleum and natural gas production or also from gasification of waste, in particular plastic waste. This can lead to a significant reduction in the carbon dioxide footprint of (petro)chemical installations.

Existing technologies such as steam cracking, methane-to-olefin or methane-to-propylene methods always produce ethylene and propylene only in a certain ratio, which can only be adjusted to a limited extent depending on the feedstock by adapting the process parameters (e.g. temperature, residence time, etc.). Here, the invention allows significant improvements and allows a targeted adjustment or focusing on a target olefin such as propylene.

Olefin metathesis, which allows flexible conversion of ethylene into propylene, proceeds nowadays from the olefins ethylene and 2-butene, which are converted to propylene. This is in principle an equilibrium reaction, and this technology was originally developed for the reverse reaction, i.e. of propylene to ethylene and butene. The 2-butene required can be obtained, for example, from the selective dimerization of ethylene or as a fraction from the production of linear α-olefins, likewise based on the oligomerization of ethylene. It is likewise possible to use 1-butene, which can be isomerized. Likewise, the use of higher internal linear olefins is also known, in which case corresponding reaction cascades ultimately always give propylene as the target product.

Although the technology of olefin metathesis can be regarded as being mature and established, it remains a major disadvantage that only high-quality and high-cost reagents are used. Olefin metathesis therefore usually serves in particular to enable flexible balancing of ethylene and propylene production and demand at a specific location or system. In contrast to olefin metathesis, the invention is advantageous because it can proceed from simple, inexpensive starting products such as customary feeds for a steam cracking method and methane.

Apart from so-called MTO or MTP methods (value chain: methane, synthesis gas, methanol, propylene), no direct route from methane or natural gas to propylene existed hitherto. By way of example, the Lurgi MTP method can be cited, however, it produces approximately 20-30% of gasoline-like compounds as a low-value by-product. The invention avoids this.

In contrast to conventional methods, in the context of the invention, significantly smaller amounts of carbon dioxide are produced, on the one hand due to the reduced firing, since this is not necessary or is only necessary to a lesser extent, in contrast to endothermic processes such as (pure) steam cracking and propane dehydrogenation, and on the other hand since no carbon dioxide is formed as a by-product, for example as in oxidative dehydrogenation.

In a preferred embodiment, the invention significantly simplifies the removal of by-products such as hydrogen, methane, carbon monoxide and carbon dioxide and further hydrocarbons in the steam cracking and of carbon monoxide and carbon dioxide in the oxidative dehydrogenation or makes a separate separation superfluous.

Further advantages of the invention result in preferred embodiments, in particular with regard to the purification and fractionation. Known routes for olefin production often require complex, in particular cryogenic separation, for example ethane/ethylene for steam cracking and oxidative dehydrogenation, of hydrocarbons having two and three or having three and more carbon atoms for steam cracking, and of propane/propylene for steam cracking and propane dehydrogenation. In a preferred embodiment, the invention makes possible in particular an integration of process steps, in particular of particularly complex steps such as cryogenic separations (e.g. in C1/C2 separations, C2/C3 separations, C3/C4 separations and so-called C2 and C3 splitters). In particular, the virtually quantitative separation of a fraction containing methane and optionally lighter compounds or a fraction containing ethane and optionally lighter compounds from higher hydrocarbons is usually required in the established processes and requires corresponding (cryogenic) apparatuses. In a preferred embodiment, the invention facilitates these steps.

The invention, to achieve the stated advantages, proposes a process for producing propylene in which a first material stream rich in ethylene is provided using a steam cracking method and a fractionation, and in which a second material stream containing carbon monoxide and hydrogen is provided using a synthesis gas production method.

The provision of the first and second material streams can comprise any separation and processing steps, in particular which are typically associated with the steam cracking method or the synthesis gas production method, for example hydrogenation and fractionation steps of known type or a separation of certain components. In principle, the steam cracking and the synthesis gas production methods can be carried out in any manner known from the prior art and with any feedstocks and reaction conditions. The first material stream rich in ethylene can contain the ethylene in particular in a content of more than, for example, 90 or 95 or 99 mole percent or in a defined purity. For example, it can be what is known as “polymer grade” ethylene, as can usually be obtained by steam cracking and subsequent fractionation. The second material stream can also comprise carbon monoxide and hydrogen, in particular in a corresponding content.

For example, the steam cracking method can be optimized for the production of ethylene and can be operated with corresponding reaction conditions and feedstocks. Particularly advantageous examples are explained below. In particular, the steam cracking method and the synthesis gas production method can also be operated in a coordinated manner with one another, in particular for adapting their respective products in a suitable stoichiometric ratio to one another.

For example, the synthesis gas production method can comprise a gasification of any feedstocks, for example plastic waste or the like. Production of synthesis gas from gaseous feedstocks, in particular methane, but also by using steam reforming, for example, is also possible. So-called dry or carbon dioxide reforming can also take place, for example. For synthesis gas production, it is possible in principle to use all methods on the proviso they provide an advantageous hydrogen-carbon monoxide ratio (ideally 1:1 only for the hydroformylation and 2:1 for the overall process with hydroformylation and hydrogenation, if no hydrogen is available from another source). Accordingly, the term synthesis gas production is used here, which is intended to comprise, in particular, steam reforming, dry or carbon dioxide reforming, partial oxidation and gasification methods, and is therefore used as an encompassing definition. Likewise, the synthesis gas production according to the invention can optionally include further steps such as a water gas shift for adjusting the hydrogen-carbon monoxide ratio or a purification (e.g., amine scrubbing, in particular Rectisol scrubbing).

In the context of the invention, at least a part of the ethylene from the first material stream is reacted with at least a part of the carbon monoxide and the hydrogen from the second material stream using a hydroformylation to obtain a third material stream of propanal. At least a part of the propanal in the third material stream is converted to propylene in the context of the invention. The ethylene is provided by means of the steam cracking method in a first component mixture; the propylene is provided in a second component mixture.

Advantageously, the first material stream is obtained here from a fractionation to which the first component mixture is subjected and which is referred to here as a “first” fractionation. Further material streams are advantageously formed in the first fractionation. The further material streams can in particular also comprise a pure propylene stream which has propylene formed in the steam cracking method.

In different embodiments of the invention, at least a part of the second component mixture is subjected either to one or more fractionation steps of the mentioned first fractionation, or to a separate fractionation, referred to here as “second” fractionation, in each case to obtain a pure propylene stream.

Accordingly, it is thus also possible according to the invention to provide one, two or more fractionations with any separation steps, which in a suitable manner each obtain the propylene from the first, from the second and/or from a combined component mixture as one or more pure propylene streams. A joint fractionation which can have two or more separate separation steps for providing a plurality of pure propylene streams can also be used in a corresponding manner. When a joint fractionation is used, any fractionation steps can be present several times, in particular in parallel, and, together or separately from one another, can be fed with material streams from the respective methods.

In addition to fractionation steps which are specifically used for providing the first material stream or the pure propylene stream(s), the fractionation can comprise further fractionation steps of any kind. The fractionation steps can comprise, for example, demethanization, deethanization, depropanization, debutanization, a separation between hydrocarbons having two (and possibly fewer) carbon atoms and three (and possibly more) carbon atoms from one another, a separation of different hydrocarbons having two carbon atoms (in particular of ethane and ethylene, so-called C2 splitters) from one another and/or a separation of different hydrocarbons having three carbon atoms (in particular of propane and propylene, so-called C3 splitters) from one another, which can be carried out in any sequence. Hydrogenation of certain components can also be provided upstream or downstream of any desired fractionation steps. The first material stream is provided in particular in a corresponding separation of different hydrocarbons having two carbon atoms, but one or more pure propylene streams, by contrast, can be provided in a corresponding separation of different hydrocarbons having three carbon atoms. As mentioned, these separations can be present one or more times and grouped in any desired separation sequences.

Particularly preferred is an embodiment in which at least a part of the first and at least a part of the second component mixture are subjected to a joint fractionation, i.e. the mentioned first fractionation, to obtain the first material stream and a joint pure propylene stream. In this case, a further fractionation is not necessarily present.

The invention makes it possible, by means of the measures proposed according to the invention, to convert ethylene into propylene according to demand using previously materially practically unused methane or other suitable carbon sources. This conversion can take place for the entire ethylene product stream of a steam cracking method or else only for a partial stream of the ethylene product. In total and in the case of idealized consideration, one molecule of propylene is obtained from one molecule of ethylene and one further carbon atom. The further carbon atom can originate, depending on the method for producing synthesis gas, from methane (in particular in steam reforming and partial oxidation) or from methane and carbon dioxide (in particular in dry or carbon dioxide reforming) or another source (in particular in the case of a gasification method). In principle, a (proportional) utilization of higher hydrocarbons, for example in steam reforming and partial oxidation, is also possible.

In the process proposed according to the invention, the reaction of at least a part of the propanal to propylene in particular comprises a hydrogenation to give propanol and a dehydrogenation to give propylene.

Although the basic process sequence of the hydroformylation of ethylene, hydrogenation and dehydrogenation is known in principle, the hydroformylation of ethylene up to now was hardly relevant in practice. In the context of the invention, the technologies and process steps of steam cracking including typically contained or necessary compression, purification and separation steps, etc., and synthesis gas production (steam reforming or alternative methods), hydroformylation, hydrogenation and dehydrogenation are now advantageously combined and integrated with one another, so that a particularly advantageous and efficient integrated method results through the use of the invention.

In particular, the third material stream may contain unconverted ethylene, hydrogen and carbon monoxide as light components, wherein the light components are transferred in a separation at least in part into a fourth material stream. The light components can be separated in particular in a C2/C3 separation, which is known per se and present in customary fractionation devices. Heavier components can be separated off in particular in a C3/C4 separation known per se.

In various embodiments of the invention, the separation of at least a part of the light components in the explained manner can take place either before the hydrogenation or between the hydrogenation and the dehydrogenation, for which purpose the order of the corresponding process steps can be reversed.

The fourth material stream or a part thereof can be returned to the process in particular as a recycle stream upstream of the hydroformylation. The return can in particular take place into the inlet of the hydroformylation. Depending on the demand, suitable post-compression or pressure adjustment can take place for pressure adjustment or for the compensation of pressure losses. If a (virtually or substantially) complete reaction of carbon monoxide and hydrogen is effected in the hydroformylation, which can take place by suitable adaptation of the stoichiometric ratios and method conditions, a corresponding formation of a recycle stream can also be omitted.

In order to avoid accumulation of traces and inerts, a part of the fourth material stream may be fed as a purge stream back to the synthesis gas production, if required. Under (possibly hypothetical) ideal conditions, in particular when no fractions of methane, ethane and/or carbon dioxide are present in a product stream of the hydroformylation, and no inert by-products are formed, a corresponding purge stream could also theoretically be dispensed with entirely. With a low input and/or formation of the stated by-products, the purge stream can be dimensioned so as to be correspondingly small.

As mentioned, measures known in principle for separating at least a part of the light components can be used within the scope of the invention. These can comprise in particular an adsorptive separation, an absorptive separation, a membrane separation, a separation by distillation and/or a phase separation. A “phase separation” is to be understood here to mean a pure separation of liquid phase and gas phase after cooling in a separation vessel having no typical separating devices of a rectification column.

In particular, an extractive distillation can be used, for example, in which a part of the propanol formed can be used as an extractant. In this way, in particular cryogenic conditions can be avoided. Due to the minimized equipment outlay, a pure separator in which the product of the hydroformylation (propanal) is obtained as liquid phase after cooling to moderate (non-cryogenic) temperatures can also be particularly advantageous.

In general, “non-cryogenic” conditions are to be understood here to mean conditions which comprise a temperature level of not less than ˜20° C., in particular not less than 0° C., in particular not less than cooling water temperature of 5 to 25° C. In the case of a pure phase separation, lighter molecules dissolved in the liquid phase are not disruptive, since they can be separated by the further integration as explained below and fed to a further material utilization.

In the process according to the invention, a hydrogen stream containing hydrogen is advantageously fed into the hydrogenation and is provided using a separate hydrogen source and/or is obtained using the synthesis gas production method and/or using the steam cracking method and is separated off using a hydrogen removal. For example, corresponding hydrogen can be obtained from the synthesis gas of synthesis gas production. In one alternative, the hydrogen can be from a corresponding light gas fraction which is obtained in the steam cracking method.

A material stream which is rich in propanal and depleted of the light components, for example a liquid material stream rich in propanal from a corresponding phase separation, can be conducted into the hydrogenation in the context of the invention. In the subsequent dehydrogenation of the propanol formed in the hydrogenation, water is formed. This is obtained in a product stream containing the propylene and the secondary components. In the context of the invention, this can be subjected to a water removal to obtain a partial stream containing the propylene and the secondary components and depleted of water.

If necessary, the water fraction can be used, for example, to generate process steam. A residue remaining after the water removal contains hydrocarbons (especially and predominantly the propylene) and possibly traces of carbon monoxide, hydrogen and, for example, propanal, propanol and further oxygenates and higher hydrocarbons.

The remaining residue, that is to say a partial stream which contains the propylene and the secondary components and has been depleted of water, or a subsequent stream formed from this partial stream in an optional trace removal, provided as required, can be subjected to one or more fractionations or fractionation steps of one or more fractionations already mentioned above to obtain a pure propylene fraction. For further explanations, reference is made to the above statements regarding the “first” and “second” fractionation.

In a particularly preferred embodiment, the product mixture produced in the steam cracking method is wholly or partly fed to a single (“first”) fractionation, as explained. In addition to the pure propylene fraction, the first material stream is also obtained wholly or partly. In other words, the water-depleted partial stream containing the propylene and the secondary components, or the subsequent stream formed from this partial stream in the optional trace removal, provided as required, is subjected in the form of the second component mixture to a joint (“first”) fractionation with the first component mixture obtained in the steam cracking method.

A combination of both component mixtures, if necessary also only of respective partial streams, takes place in accordance with the pressure level at a suitable point. In this way, in particular the separation and purification outlay for the production of pure propylene can be reduced. The feed of the first and second component mixture or corresponding parts thereof can take place at different points of the fractionation, depending on the composition of the component mixtures. The ethylene, i.e., the first material stream, can in particular be taken from a so-called C2 splitter, which may also be present in the steam cracking method, on the other hand, the pure propylene can be taken from a so-called C3 splitter, which may also already be present in the steam cracking method, as already mentioned. The resulting pure propylene stream can therefore comprise, when a single fractionation is used, a proportion of propylene from the steam cracking method and a proportion of propylene from the sequence of hydroformylation, hydrogenation and dehydrogenation. In this case, the invention can also comprise in particular the parallelization of individual or several assemblies, such as separations, C2 splitters and C3 splitters, if a parallelization is required due to the process management, constructability and/or scalability, for example.

In this particularly preferred embodiment of the invention, the required purifications and separations can be completely or partially run through in a joint fractionation, whereby undesired components can be separated off. A separation of propylene and any propane formed as a by-product of an overhydrogenation can take place, for example, in a C3 splitter which is present anyway. Heavier components can be removed beforehand, if present, from propane and propylene. The propane removed can in turn be made available to the steam cracking method wholly or partly as a feedstock material. Higher hydrocarbons (having, for example, four and more carbon atoms) can in this way also optionally be conducted wholly or partly as separate material streams out of the overall process and utilized, or else can again be made available to the steam cracking method wholly or partly as feedstock material. As a result, any trace products or by-products arising from the process chain of hydroformylation, hydrogenation and dehydrogenation and having four or more carbon atoms can also be fed to a material utilization. This applies analogously to further compounds with corresponding boiling behavior, e.g., in particular alcohols.

In the course of the overall process, therefore, in the ideal case, all the secondary streams obtained are materially utilized by means of a corresponding return, which represents a particular advantage of the invention.

In general, in the invention, in particular methane can be materially used in a meaningful manner, which often arises as an “accompanying gas”, for example in petroleum production, and has not been materially utilized to date. This is thus directly linked to the recovery of corresponding steam cracking feedstocks such as naphtha, so-called atmospheric gas oil (AGO) or vacuum gas oil (VGO). Even if light fractions such as ethane, propane and butane or so-called liquefied petroleum gas (LPG) are used as a steam cracking feedstock, the usually contained considerable fractions of methane must be removed beforehand. If no infeed, for example into natural gas pipelines, is possible, the methane is usually mostly burned. Although the resulting carbon dioxide is less climate-damaging than methane, it still contributes to a considerable extent to global warming. The invention avoids this by the described use of the synthesis gas production method in which the methane can be used in the proposed overall process. Alternatively, the use of synthesis gas from gasification methods enables the material utilization of waste, in particular plastic waste.

Other methods for (selective) propylene production often convert already high-quality products, as already explained above. As already mentioned, in the olefin metathesis, already high-quality products (olefins, in particular ethylene and 2-butene) are thus converted to propylene, while in the proposed method the upgrade of ethylene to propylene is effected from the use of methane (at an ideal ratio of the carbon balance with two carbon atoms from ethylene and one carbon atom from methane). So-called on-purpose methods such as propane dehydrogenation produce substantially propylene as the product, but lighter or heavier fractions which are likewise obtained here cannot be used or cannot be used selectively for the production of propylene.

The method described here, on the other hand, allows the use of all of these fractions in the steam cracking with the corresponding downstream method steps and the synthesis gas production method, as well as a demand-based and flexible adaptation of the ratio of ethylene to propylene. In this way, ethylene can thus be converted as required and cost-effectively into propylene. This results in particular advantages due to the integration of process steps and the joint use of process steps that are present and/or are to be incorporated in any case, such as compression, purification, drying and separation steps (in particular comprising a C3 splitter, which represents a considerable equipment outlay), as in embodiments of the invention. These steps have hitherto usually been connected downstream of the steam cracking method as an independent separation sequence.

The efficiency of the process can be stated as an olefin efficiency factor O_{Ef} or monomer efficiency MWG in %. It results initially as the quotient of the mass of pure propylene M_{Pure propylene} obtained via the reaction cascade of hydroformylation, hydrogenation and dehydrogenation, and the mass of ethylene m_{Ethylene} used in the hydroformylation, as follows:

O_{Ef}=MWG=100%*m_{Pure propylene}/m_{Ethylene}

Under idealized conditions (i.e., 100% conversion in each case with 100% selectivity) this value is at most 150%. The olefin efficiency factor O_{Ef} or monomer efficiency MWG can also be derived from the corresponding individual steps of the method. Each individual step (i=1 to x)—i.e., in particular the hydroformylation, a separation of light components, the hydrogenation, the dehydrogenation and a fractionation—is characterized here by a pair of conversion Xᵢ and selectivity Sᵢ, which can be optimally achieved under technical conditions. This results in the particular yield of the individual step:

Yᵢ=Xᵢ*Sᵢ.

For a separation and a fractionation, the conversion can in each case be set to 100%, since although no reaction of a reagent to form a different product occurs here, the inlet mass must be equal to the outlet mass for each component. Reagent and product are therefore identical components for separation or fractionation when considered in this way. The selectivity is then a measure here for the efficiency of the separation or fractionation. With a selectivity of 100%, the corresponding component reaches the target fraction completely, while at values less than 100% a loss into another fraction of the separation or fractionation takes place. Values less than 100% therefore quantify the corresponding loss of the component in question that does not reach the target fraction.

Firstly, the following is true for the overall yield Y_{Total} of the process based on ethylene and the reaction products further formed therefrom:

Y_{Total}=Πᵢ₌₁^{y}YᵢΠᵢ₌₁^{x}Yᵢ

If unconverted reagents are returned by recycling into the corresponding reaction step (in particular, for example, unconverted ethylene in the hydroformylation), there may be a higher conversion for the corresponding individual step; therefore, a correspondingly adjusted higher value Xᵢ or Yᵢ is then to be established for this step.

The olefin efficiency factor or monomer efficiency is dependent on the efficiency of each individual method step and can be calculated by multiplication of Y_{Total} by the molar mass ratio of propylene to ethylene:

O_{Ef}=MWG=100%*Y_{Total}*(M_{C3H6}/M_{C2H4})=100% Πᵢ₌₁^{x}Yᵢ*(M_{C3H6}/M_{C2H4})

From the corresponding individual steps and their characteristic values for Xᵢ, Sᵢ and Yᵢ, it can be deduced that the olefin efficiency factor or monomer efficiency in a preferred embodiment consisting of hydroformylation, separation of light components, hydrogenation, dehydrogenation and fractionation is at least 100%, in particular at least 115%, 125% or 130%, and up to 150%.

A comparable olefin efficiency factor O_{Ef} (metathesis) can also be calculated for the olefin metathesis, but both ethylene and butene, which are frequently obtained from the dimerization of ethylene, are to be incorporated here as high-quality olefinic reagents:

O_{Ef} (metathesis)=100%*m_{Pure propylene}/(m_{Ethylene}+m_{Butene})

By definition, the olefin efficiency factor O_{Ef} (metathesis) for olefin metathesis is therefore always <100% and only reaches 100% in an ideal case. The olefin efficiency factor or monomer efficiency O_{Ef}>100% illustrates the particular advantage as well as the practical benefit of the invention, since, according to the invention, values for the olefin efficiency factor O_{Ef} higher than 100% are particularly preferably obtained.

In a particularly advantageous embodiment of the method according to the invention, carbon dioxide can be fed into the synthesis gas production method, which is suitably configured for this purpose. This can take place in particular in the case of steam or carbon dioxide or dry reforming. This achieves an increased proportion of carbon monoxide in the outlet stream of the reformer. Such a variant appears to be advantageous in particular in the case of steam cracking methods with light feedstock (ethane, propane, butane, LPG), since here in any case a significant amount of hydrogen is obtained in the cracking process and can be provided as a separate hydrogen stream for the hydroformylation and/or hydrogenation. However, carbon dioxide can also originate from an external source (pure or in a mixture with methane) or else from carbon dioxide scrubbing, i.e., typically amine scrubbing. In particular, an extraction from the exhaust gas/flue gases as are produced during heating with burners, can be advantageous here in order to minimize the carbon dioxide footprint of an integrated overall process. It is likewise thus possible to use carbon dioxide as a by-product in the process gas of the steam cracking method.

In a further advantageous embodiment, the process comprises carrying out an electrical heating in the steam cracking method and/or synthesis gas production method, and feeding methane into the synthesis gas production method, the methane being formed in the steam cracking method. In other words, methane which originates wholly or partly from a corresponding fraction from the steam cracking method can be directly moved into the synthesis gas production. In this way, a corresponding material stream, which is otherwise used for firing the furnaces, can be utilized materially.

In addition, the (partly) electrical operation of drives of, for example, pumps, compressors and turbines is possible in order to minimize or completely avoid an additional steam demand of the overall process. In this way, a further minimization of the emission of carbon dioxide is possible, i.e., no carbon-containing raw material has to be burnt for steam generation, but this carbon-containing raw material can be partly or completely guided into a suitable step for synthesis gas production, provided according to an advantageous embodiment of the invention.

In the case of a gasification method for synthesis gas production, the complete or partial use of waste plastic (“plastic recycling”) provides beneficial recycling of such waste in the sense of a sustainable circular economy.

In one embodiment of the invention, the process can also comprise forming the first material stream, which contains hydrogen, using a tail-end hydrogenation associated with the steam cracking method. A method variant in which the C2 hydrogenation of the steam cracker is effected as so-called tail-end hydrogenation appears particularly advantageous. Tail-end hydrogenation denotes a hydrogenation of a formed fraction and not of a total mixture present upstream. The hydrogenation is effected for the reaction of acetylene(s). In the context of the invention, an excess can be tolerated after the tail-end hydrogenation, without an additional fine purification of the first material stream being required. This advantage comes into effect fully when all ethylene from the cracker is utilized in the method according to the invention, since no partial stream then has to be purified as an independent end product. The method according to the invention requires hydrogen and carbon monoxide in the hydroformylation anyway.

The mentioned water-depleted partial stream containing the propylene and the secondary components, or a subsequent stream which is formed from this partial stream in an optional trace removal, provided as required, and which is obtained at low pressure can be conducted, for example, before a corresponding compressor stage in a so-called crude gas hydrogenation of the steam cracking method at a suitable pressure level. It can in particular be the second stage. Instead of performing a separate water depletion, this can likewise take place in a fractionation which is associated with the steam cracking method and is typically carried out upstream of the crude gas compression. This results in a corresponding pressure loss after the dehydrogenation, which, however, already typically takes place at a relatively low pressure level.

At any point, waste heat from the synthesis gas production can be used for the steam cracking method, in particular for preheating and/or steam generation or provision of low-pressure steam.

In principle, the ethylene used can also originate entirely or partly from any desired source which provides ethylene in corresponding purity (for example ethanol dehydrogenation, oxidative dehydrogenation, MTO/MTP methods). Due to the possible integration with the preceding method, however, particular relevance results for methods in which a separation of the C3 fraction is likewise included in the preceding olefin production (e.g., steam cracking, MTO/MTP methods).

The invention also relates to an installation for the production of propylene, with a steam cracking device and a fractionation which is configured to carry out a steam cracking method and to provide an ethylene-containing first material stream, with a synthesis gas producing device which is configured to carry out a synthesis gas production method and to provide a second material stream containing carbon monoxide and hydrogen, and with a hydroformylation device which is configured to react at least a part of the ethylene from the first material stream with at least a part of the carbon monoxide and of the hydrogen from the second material stream to form propanal using a hydroformylation to obtain a third material stream, wherein the installation is further configured to provide the ethylene by means of the steam cracking method in a first component mixture, to react at least a part of the propanal in the third material stream to form propylene, and in the process to provide a second component mixture containing the propylene. As already explained with respect to the method proposed according to the invention, it can be provided to subject at least a part of the first and at least a part of the second component mixture to one or more fractionations or fractionation steps to obtain the first material stream and one or more pure propylene streams.

A corresponding installation is configured in particular for carrying out a method as explained above in different embodiments. Therefore, reference can be made at this juncture to the corresponding explanations, which also relate to the installation proposed according to the invention.

The invention will be explained in more detail below with reference to specific examples and the accompanying drawings, which illustrate preferred embodiments of the invention.

### EXAMPLES

As already stated, different variants (steam reforming, dry or carbon dioxide reforming, partial oxidation, gasification methods) and also combinations in particular of steam reforming and dry reforming are suitable for the synthesis gas production. In an idealized manner, the following equations I, III and IV describe the respective conversion of methane, while equations II, IV and VI describe the respective conversion of ethane by way of example. In addition, the resulting H₂/CO ratio is specified.

### Steam Reforming:

CH₄+H₂O→CO+3 H₂ H₂/CO=3:1 (I)

C₂H₆+2 H₂O→2 CO+2 H₂ H₂/CO=2.5:1 (II)

### Dry Reforming:

CH₄+CO₂→2 CO+2 H₂ H₂/CO=1:1 (III)

C₂H₆+O₂→2CO₂→4 CO+3 H₂ H₂/CO=0.75:1 (IV)

### Partial Oxidation (PDX):

2 CH₄+O₂→O₂CO+4 H₂ H₂/CO=2:1 (V)

C₂H₆+O₂→2 CO+3 H₂ H₂/CO=1.5:1 (VI)

In addition to these reactions I to VI, further reactions can also take place, such as water gas shift and reverse water shift according to the equations VIIa and VIIb or, in particular in the case of the partial oxidation, a total oxidation into carbon dioxide and water. This then leads to deviations from the ideal behavior outlined here and shifts in the ratio of hydrogen to carbon monoxide.

### Water Gas Shift:

CO+H₂O→H₂+CO₂ (VIIa)

### Reverse Water Gas Shift:

H₂+CO₂→CO+H₂O(VIIb)

Under real conditions, corresponding deviations thus result. The reforming of methane does not provide, for example, exactly a ratio of hydrogen to carbon monoxide of 3:1, because this ratio is shifted precisely by the shift reaction VIIa. Here, the characteristic S_{N}, the so-called stoichiometric modulus or the stoichiometric number, is helpful.

$${S}_{N} := \frac{{x}_{{H}_{2}} - {x}_{{CO}_{2}}}{{x}_{CO} + {x}_{{CO}_{2}}}$$

The stoichiometric number is invariant with the shift reaction: If the shift reaction was completely shifted to the side of the carbon monoxide, the product would be a gas without carbon dioxide and with a ratio of hydrogen to carbon monoxide corresponding to the stoichiometric number. The real ratios are therefore better described by the stoichiometric number.

The reforming of pure methane without carbon dioxide and without higher hydrocarbons in the reaction feedstock always provides, for example, exactly a stoichiometric number of 3. When carbon dioxide is returned, for example through a part of the aforementioned fourth material stream (stream 6 in the subsequent figures), this carbon dioxide can then be returned to the synthesis gas production and, in particular in reforming and dry reforming, can be converted according to equations III and IV.

If the individual reactions of hydroformylation (equation IIX), hydrogenation (IX) and dehydrogenation (X) are now considered, the corresponding hydrogen and carbon monoxide demand is obtained as follows:

### Hydroformylation:

C₂H₄+H₂+CO→C₃H₆O Demand for H₂/CO=1:1/S_{N}=1:1 (IIX)

### Hydrogenation:

C₃H₆O+H₂→C₃H₇OH Demand for only 1 equivalent H₂ (IX)

### Dehydrogenation:

C₃H₇OH→C₃H₆+H₂O No demand for H₂ and CO (X)

For an ideal overall reaction of the integrated process (hydroformylation, hydrogenation and dehydrogenation), the following gross equation XI and the total demand given below (tot. dem.) are given:

C₂H₄+2 H₂+CO→C₃H₆+H₂O tot. dem. H₂/CO=2:1/S_{N}=2:1 (XI)

Accordingly, one of the aforementioned methods for synthesis gas production can be advantageous depending on the availability of further hydrogen. If sufficient other hydrogen is available, dry reforming is particularly advantageous since this provides high yields of carbon monoxide and an ideal synthesis gas stream for the hydroformylation. The partial oxidation of methane appears advantageous if both the carbon monoxide demand and the total demand for hydrogen can be covered thereby.

Pure steam reforming of methane initially provides an excess of hydrogen, which, however, can be converted again into carbon monoxide by a reverse water gas shift according to equation VIIb. In this case, carbon dioxide is also advantageously reacted and water is produced as a further reaction product.

According to equations II, IV and VI, the ratio of hydrogen to carbon monoxide decreases in each case when higher hydrocarbons are fed in into the synthesis gas production. However, this does not represent a problem since in any case too much hydrogen is produced in most methods than is required in the method according to the invention. An adjustment as required is always possible in this case by means of water gas shift or reverse water gas shift according to equations VIIa and VIIb.

Overall, however, under real conditions, a method for synthesis gas production which combines steam reforming and dry reforming and provides the stoichiometry as required appears to be particularly suitable. In contrast to a partial oxidation or gasification, the additional advantage of the integrated material utilization of carbon dioxide is provided here.

Overall, it is apparent from equation XI that the propylene formed is formed from ethylene and carbon monoxide. Since this carbon monoxide is formed as mentioned from methane or even proportionally from carbon dioxide, a clear increase in value in the process chain is obtained starting from ethylene using methane and/or carbon dioxide otherwise hardly or not materially used.

If reference is made below to process or method steps, these are also to be understood to cover the equipment used in each case for these method steps (in particular, for example, reactors, columns, scrubbing devices, etc.), even if this is not expressly referred to. In general, the explanations relating to a process apply to a corresponding installation in the same way in each case.

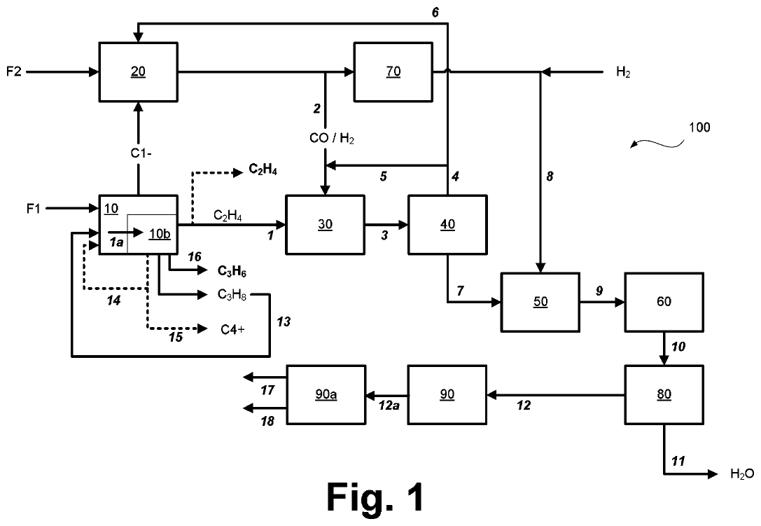

FIG. 1 illustrates a process according to a preferred embodiment of the invention in the form of a schematic flowchart, which process is denoted overall by 100.

In the process 100, a first material stream 1 which is rich in ethylene is provided in a steam cracking method 10 using a fractionation 10b. The fractionation 10b is illustrated only in highly simplified form as a dashed block and in particular by means of some material streams obtained in the process, which are shown in the form of corresponding flow arrows. A product mixture, denoted by 1a, of the steam cracking method 10 is fed to the fractionation 10b and is also referred to here as a “first component mixture”.

For example, in the fractionation steps of the fractionation 10b, a fraction denoted C4+ which contains hydrocarbons having four and more carbon atoms can be provided in the form of a material stream 15 and returned wholly or partly in the form of a material stream 14 into the steam cracking method 10. The same applies to a propane fraction C₃H₈, which can be returned in the form of a material stream 13. A first pure propylene fraction is provided as product fraction 16. A suitable feedstock stream F1 is fed to the steam cracking method.

For further explanations, in particular with regard to the performance of the steam cracking method 10 and the formation of the fractions mentioned, reference can be made to conventional technical literature such as the article “Ethylene” in Ullmann's Encyclopedia of Industrial Chemistry, for example the publication of 15 Apr. 2009, DOI: 10.1002/14356007.a10_045.pub2.

In a synthesis gas production method 20, which can comprise, in particular, steam reforming and dry reforming, a second material stream 2 containing carbon monoxide and hydrogen is provided using a second feedstock stream F2 and can be provided from a crude product stream, in particular in the context of a separation of hydrogen explained below.

For possible synthesis gas production methods 20, reference is made to the above explanations and, for example, also the article “Gas Production” in Ullmann's Encyclopedia of Industrial Chemistry, for example the publication of 15 Dec. 2006, DOI: 10.1002/14356007.a12_169.pub2.

In the example illustrated here, at least a part of the ethylene from the first material stream 1 is reacted with at least a part of the carbon monoxide and of the hydrogen from the second material stream 2 to form propanal using a hydroformylation 30 to obtain a third material stream 3. The third material stream 3 typically also contains unconverted components of the material streams 1 and/or 2. At least a part of the propanal in the third material stream 3 is reacted to obtain a material stream 7 by use of a hydrogenation 50 to form propanol (material stream 9), and the latter is reacted to give the propylene using a dehydrogenation 60 to obtain a material stream 10.

At least a part of the fourth material stream 4 can be returned into the process 100 as a recycle stream 5 upstream of the hydroformylation 30. As mentioned, the return of at least a part of the fourth material stream 4 can comprise a post-compression. Apart of the fourth material stream 4 can also be returned to the synthesis gas production method 20 as a purge stream 6.

The separation 40 of at least a part of the light components can be carried out in different ways, in the simplest case, as mentioned, in the form of a deposition of a liquid fraction.

A hydrogen stream 8 which is obtained using the synthesis gas production method 20 is conducted into the hydrogenation 50. The hydrogen stream is separated using a hydrogen removal 70, for example comprising a pressure swing adsorption. Additional hydrogen H₂ may also be provided and supplied to the method using a separate hydrogen source, if required.

In the dehydrogenation 60, a product stream containing water, the propylene and secondary components, the material stream 10 already mentioned above, is formed and is subjected to a water removal 80 to obtain a partial stream 12 containing the propylene and the secondary components and depleted of water. In the water removal, a water stream 11 is formed, which can be used, for example, to provide process steam.

The water-depleted partial stream 12 containing the propylene and the secondary components or a subsequent stream 12a formed therefrom in an optional trace removal 90 is fed to a further fractionation 90a, in which a second pure propylene product fraction 17 and a further material stream 18, which in particular contains propane and/or higher-boiling components, are obtained.

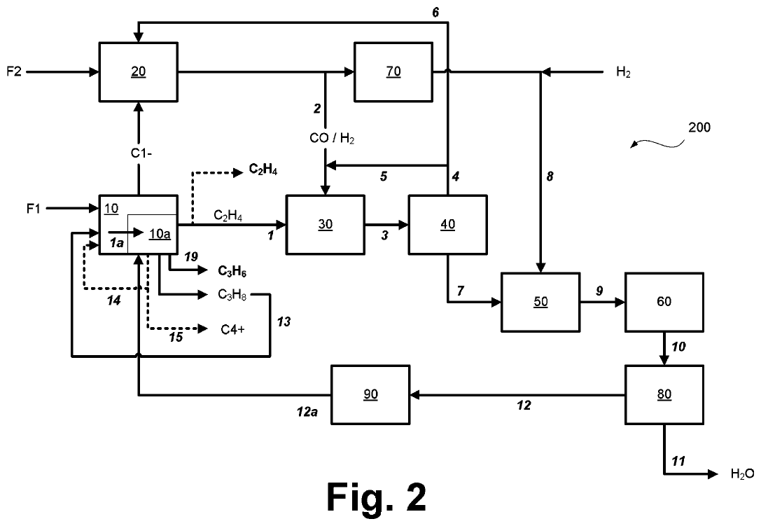

FIG. 2 illustrates a process according to a further preferred embodiment of the invention in the form of a schematic flowchart, which process is denoted overall by 200. In principle, the explanations previously described for FIG. 1 and the process 100 apply. Deviations are explained below.

The partial stream 12, which contains the propylene and the secondary components and has been depleted of water, or the subsequent stream 12a formed therefrom in an optional trace removal 90 is fed here to a fractionation 10a, to which, as in the process 100 according to FIG. 1, a product mixture produced in the steam cracking method 10 is also subjected. The fractionation 10a can be configured as previously or can be adapted to the specific requirements of the additional processing of the material stream 12 or of the subsequent stream 12a. In this way, a joint pure propylene product fraction 19 can be formed in the fractionation 10a. The pure propylene fraction 19 thus comprises propylene from the steam cracking method 10 and the process chain of hydroformylation 30, hydrogenation 50 and dehydrogenation 60.

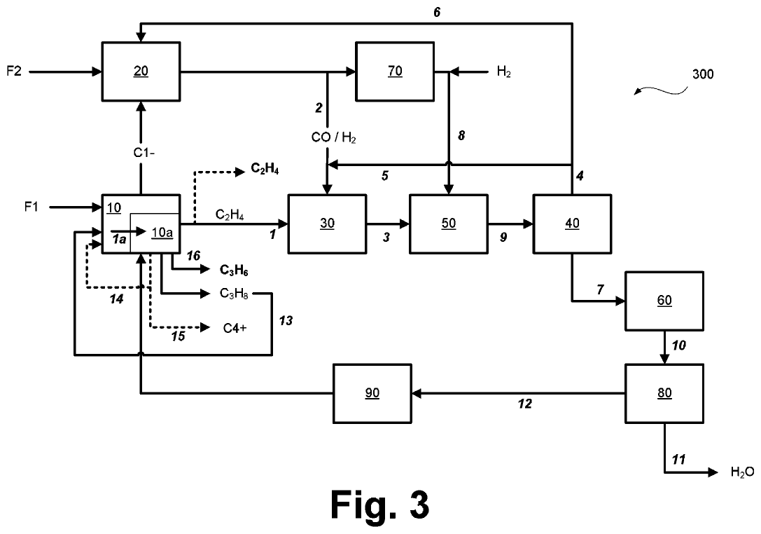

FIG. 3 illustrates a process according to a further preferred embodiment of the invention in the form of a schematic flowchart, which process is denoted overall by 300. Here too, as in the method 200 according to FIG. 2, a joint fractionation 10a is provided. Alternatively, however, the fractionation 10b shown in FIG. 1 for process 100 can also be provided. As illustrated here, separation 40 takes place here between the hydrogenation 50 and the dehydrogenation 60, which represents a particularly preferred variant. For the material streams and the designations, reference is made to the above statements. Although material streams from the separation 40 are denoted by 7 and those from the hydrogenation by 9, as before, it is understood that they have a different composition due to the different sequence. The designation was maintained on account of the type of provision.

1. A process for producing propylene, comprising:
providing a first material stream using a steam cracking method and one or more fractionations, the first material stream being rich in ethylene;
providing a second material stream containing carbon monoxide and hydrogen using a synthesis gas production method;
reacting at least a part of the ethylene from the first material stream with at least a part of the carbon monoxide and the hydrogen from the second material stream to form propanal using a hydroformylation to obtain a third material stream (3); and
converting at least a part of the propanal in the third material stream to propylene;
wherein:
the ethylene is provided by means of the steam cracking method in a first component mixture; and
the propylene is provided in a second component mixture.

2. The process according to claim 1, further comprising:
subjecting at least a part of the first component mixture to a first fractionation to obtain the first material stream; and
subjecting at least a part of the second component mixture to one or more fractionation steps of the first fractionation or a second fractionation to obtain a pure propylene stream.

3. The process according to claim 1, in which the reaction of at least a part of the propanal to form the propylene comprises a hydrogenation to give propanol and a dehydrogenation to give propylene to obtain the second product mixture.

4. The process according to claim 3, in which the third material stream contains unreacted ethylene, hydrogen and carbon monoxide as light components, wherein the light components are transferred in a separation at least in part into a fourth material stream.

5. The process according to claim 4, in which the separation of at least a part of the light components is carried out before the hydrogenation or between the hydrogenation and the dehydrogenation.

6. The process according to claim 4, in which at least a part of the fourth material stream is returned into the process as a recycle stream upstream of the hydroformylation.

7. The process according to claim 5, in which the return of at least a part of the fourth material stream comprises a post-compression.

8. The process according to claim 6, in which a part of the fourth material stream is returned to the synthesis gas production method.

9. The process according to claim 6, in which the separation of at least a part of the light components comprises an adsorptive separation, an adsorptive separation, a membrane separation, a separation by distillation and/or a phase separation.

10. The process according to claim 3, further comprising feeding a hydrogen stream containing hydrogen into the hydrogenation, the hydrogen stream being provided using a separate hydrogen source and/or is obtained using the synthesis gas production method and/or using the steam cracking method and is separated using a hydrogen removal.

11. The process according to claim 3, further comprising forming in the dehydrogenation a product stream containing water, propylene and secondary components, and subjecting the product stream to a water removal to obtain the partial stream containing propylene and also the secondary components and depleted of water.

12. The process according to claim 11, further comprising subjecting the partial stream or a subsequent stream formed therefrom in a trace removal at least partly as the second component mixture to the first or second fractionation.

13. The process according to claim 1, further comprising feeding carbon dioxide into the synthesis gas production method.

14. The process according to claim 1, in which electrical heating is performed in the steam cracking method and/or in the synthesis gas production method, and in which methane formed in the steam cracking method is fed into the synthesis gas production method.

15. The process according to claim 1, in which one or more pumps, one or more compressors and/or one or more turbines are used that are at least partially electrically driven.

16. The process according to claim 1, in which the first material stream is formed using a tail-end hydrogenation and contains hydrogen.

17. The process according to claim 1, in which the olefin efficiency factor and/or monomer efficiency is at least 100%, in particular at least 115%, 125% or 130%, and up to 150%.

18. An installation for the production of propylene, comprising:
a steam cracking device and one or more fractionations, configured to carry out a steam cracking method and to provide an ethylene-containing first material stream;
a synthesis gas producing device configured to carry out a synthesis gas production method and to provide a second material stream containing carbon monoxide and hydrogen; and
a hydroformylation device configured to react at least a part of the ethylene from the first material stream with at least a part of the carbon monoxide and of the hydrogen from the second material stream to form propanal using a hydroformylation to obtain a third material stream;
wherein the installation is configured to provide the ethylene by means of the steam cracking method in a first component mixture, to react at least a part of the propanal in the third material stream to form the propylene and in the process to provide a second component mixture containing the propylene.

19. The process according to claim 5 in which at least a part of the fourth material stream is returned into the process as a recycle stream upstream of the hydroformylation.

20. The process according to claim 7, in which the separation of at least a part of the light components comprises an adsorptive separation, an adsorptive separation, a membrane separation, a separation by distillation and/or a phase separation.
